(19) United States
(12) Patent Application Publication
(10) Pub. No.: US 2011/0060247 A1
(43) Pub. Date: Mar. 10, 2011

(54) METHODS AND APPARATUS FOR MEASURING BONE LENGTHS

(75) Inventors: Randall Payne, Madison, WI (US); Mathew Frank, Verona, WI (US)

(73) Assignee: GENERAL ELECTRIC COMPANY, Schenectady, NY (US)

(21) Appl. No.: 12/557,280

(22) Filed: Sep. 10, 2009

(51) Int. Cl. A61B 5/103

(52) U.S. Cl. 600/587

(57) ABSTRACT

Methods and apparatus measuring bone lengths are provided. The methods include acquiring bone image information from a dual-energy x-ray scan of a subject and generating a dual-energy image using the acquired bone information. The methods further include determining a length of at least one bone of the subject using the dual-energy image.

### BACKGROUND OF THE INVENTION

The subject matter disclosed herein relates generally to medical diagnostic imaging systems, and more particularly to diagnostic imaging systems that acquire bone images.

Bone length and limb measurements, particularly of the long bones (e.g., bones that are longer than wide) in the human body, are commonly used to determine potential diseases or conditions that can cause bone growth problems, such as disproportional limb growth. These measurements are also used to monitor disproportionate bone growth or limb length discrepancies. Disproportional bone lengths can be indicative not only of bone growth problems, such as pediatric bone growth problems, but can cause physical pain, for example, back pain resulting from one leg being longer than another leg. The disproportional bone lengths often cannot be determined from an external examination. Additionally, when assessing the growth of children, measurements other than external height measurements are useful to assess potential bone problems and growth issues.

Conventional methods to estimate bone length include the use of anthropometers that estimate bone length from external measurements. These devices are often not particularly accurate and susceptible to operator error. Radiographic methods for measuring bone length are also known and include the use of radiographs and scanograms to generate bone images from which bone lengths are determined. These radiographic methods must compensate for magnification errors and also use a relatively high ionizing radiation dose. High x-ray dose can be an issue particularly when monitoring pediatric growth, for example, monitoring growth over time. Also, the radio-opaque rulers used to reduce magnification errors can be difficult to accurately read, leading to measurement errors. Moreover, parallax errors in these conventional imaging systems can lead to problems or inaccuracies when determining bone length from the bone images.

### BRIEF DESCRIPTION OF THE INVENTION

In accordance with one embodiment, a method for measuring bone length is provided. The method includes acquiring bone image information from a dual-energy x-ray scan of a subject and generating a dual-energy image using the acquired bone information. The method further includes determining a length of at least one bone of the subject using the dual-energy image.

In accordance with another embodiment, a method for determining bone length is provided. The method includes acquiring image information with a bone densitometer using a dual-energy full body scan. The image information includes bone content information and soft tissue information. The method further includes identifying endpoints of at least one bone using images formed from the image information and calculating a distance between the endpoints to determine a length of the at least one bone.

In accordance with yet another embodiment, a diagnostic imaging system is provided that includes a dual-energy x-ray bone densitometer configured to acquire bone information from a dual-energy imaging scan of a subject. The diagnostic imaging system further includes a bone length measurement module configured to determine a length of at least one bone of the subject using the bone information from the dual-energy imaging scan.

### BRIEF DESCRIPTION OF THE DRAWINGS

FIG. 1 is a block diagram of a dual-energy x-ray imaging system formed in accordance with various embodiments illustrating a full body scan.

FIG. 2 is a flowchart of a method for acquiring bone image information to determine the lengths of bones in accordance with various embodiments.

FIG. 3 is a block diagram of a dual-energy x-ray imaging system formed in accordance with various embodiments illustrating joint region scans.

FIG. 4 is an image from a full body dual-energy x-ray imaging scan performed by the system of FIG. 1.

FIG. 5 is an image from a joint region dual-energy x-ray imaging scan performed by the system of FIG. 3.

FIG. 6 is an enlarged view of a potion of the image of FIG. 4.

FIG. 7 is a graph showing the correlation of bone length measurement using dual-energy imaging according to various embodiments to caliper ruler measurements.

### DETAILED DESCRIPTION OF THE INVENTION

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware circuitry. One or more of the functional blocks (e.g., processors or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Exemplary embodiments of dual-energy x-ray systems and methods for measuring bone lengths are described in detail below. In particular, a detailed description of an exemplary dual-energy x-ray system will first be provided followed by a detailed description of various embodiments of methods and systems for measuring bone lengths.

At least one technical effect of the various embodiments of the systems and methods described herein is to measure bone lengths using a dual-energy x-ray system. In some embodiments a single dual-energy x-ray scan, and more particularly, a single body scan is used to acquire image information for a number of different bones, from which bone lengths are determined.

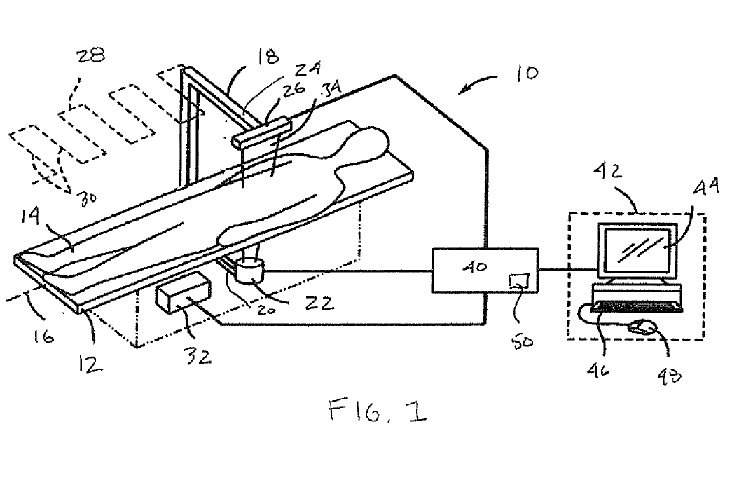

FIG. 1 is a block diagram of an exemplary dual-energy x-ray system, illustrated as a dual x-ray absorptiometry (DEXA or DXA) system 10, which is also referred to as dual energy bone densitometer system capable of performing bone densitometry. The system 10 constructed in accordance with various embodiments is configured to measure at least an area of a bone, a length of a bone, a bone mineral content (BMC), and a bone mineral density (BMD). The BMD is calculated by dividing the BMC by the area of the bone. During operation, two x-ray beams having different energy levels are utilized to scan an object, for example, to scan a human subject to image the bones of the human subject. The acquired images of the bones are used to calculate the length of at least some of the bones, which may include monitoring the change in length of the bones over time. The images may be generated in part from determined bone density information acquired during a dual-energy x-ray scan.

The system 10 includes a patient table 12 providing a horizontal surface for supporting a subject, for example, a patient 14 in a supine or lateral position along a longitudinal axis 16. The system 10 also includes a support member, for example, a C-arm 18. The C-arm 18 has a lower end 20 that is positioned beneath the patient table 12 to support an x-ray source 22. The C-arm 18 has an upper end 24 that is positioned above the patient table 12 supporting an x-ray detector 26. The x-ray detector 26 may be fabricated, for example, as a multi-element cadmium-zinc-telluride (CZT) detector providing for energy discrimination. The x-ray source 22 and the x-ray detector 26 may be moved in a raster pattern 28 so as to trace a series of transverse scans 30 of the patient 14 during which dual energy x-ray data is collected by the x-ray detector 26. The raster motion is produced by actuators (not shown) under control of a translation controller 32. During operation, the x-ray source 22 produces a fan beam 34 having a plane that is parallel to the longitudinal axis 16. The raster pattern 28 is adjusted such that there is some overlap (e.g., slight overlap of 10 percent) between successive scan lines of the fan beam 34.

The x-ray source 22, the x-ray detector 26, and the translation controller 32 communicate with, and are under the control of, a computer 40 which may include both dedicated circuitry and one or more processors having the ability to execute a stored program. In the exemplary embodiment, the computer 40 also includes a bone length measurement module 50. The module 50 utilizes the scan data or the scanned image to determine the bone lengths of at least some of the bones of a scanned skeleton of the patient 14. During operation, the module 50 directs the dual-energy imaging system 10 to acquire a full body (or total body scan) or smaller region scans, from which certain bones are identified and measured, for example, by identifying bone landmarks (e.g., bone plates). The locations of the landmarks may be determined automatically, manually or semi-automatically, for example, with an operator adjusting or fine tuning automatically generated landmarks of defining points.

The module 50 then utilizes the scan data or scan image(s) to select certain points of interest that correspond to determined endpoints of bones, such as the landmarks. In various embodiments, the end points of a predetermined set of long bones are determined as described in more detail below. It should be noted that different kernels may be used to identify the bone endpoints, for example, using a gap or decrease is diameter of the bone that corresponds to a bone endplate. An operator is then able to adjust the landmarks identifying the bone endpoints, which are also displayed with a graphical overlay showing the entire length of the identified bone.

Referring again to FIG. 1, the computer 40 communicates with a terminal 42 including a display 44, a keyboard 46, and a cursor control device such as a mouse 48 allowing for operator input and the output of text and images to the operator. In some embodiments, the computer 40 is located remotely from the workstation 42. Optionally, the computer 40 may form a portion of the workstation 42. The computer is adapted to perform one or more processing operations. The acquired bone information, for example, image and density information may be processed and displayed in real-time during a scanning session as the data is received. Additionally or alternatively, the data may be stored temporarily in a memory device on the computer 40 during a scanning session and then processed and displayed in an off-line operation. The information may also be stored in a long-term storage device (e.g., hard-drive or server) for later access, such as during a follow-up scan of the same patient and useful to monitor the progress of bone growth or change in bone lengths. The display 44 includes one or more monitors that present patient information, including the scanned image and the bone length images to the operator for diagnosis and analysis. The displayed images may be modified and the display settings of the display 44 also manually adjusted using the keyboard 46, the mouse 48, or a touch screen icon on the display itself.

During operation, the system 10 is configured to operate in either a dual energy x-ray mode or a single energy x-ray mode. In the single energy mode, the x-ray source 22 emits x-rays at a narrow band of energies of a few keV and in the diagnostic imaging range of approximately 20-150 keV. In the dual-energy mode, the x-ray source 22 emits radiation at two or more bands of energy emitted simultaneously or in rapid succession. The x-ray source 22 may also be configured to emit a single broadband energy of more than a few keV over the diagnostic imaging range. The system 10 may be switched between the dual energy mode and the single energy mode by increasing or decreasing the x-ray source 14 voltage and/or current. The system may also be switched between the dual energy mode and the single energy mode by removing or adding a K-edge filter.

The x-ray source 22 may be configured to output a fan beam of x-rays 34 as shown in FIG. 1. The x-ray source 22 may also be configured to output a pencil beam of x-rays (not shown), a cone beam of x-rays, or other configurations. In some embodiments, the module 50 controls the system 10 to operate in the single energy mode or dual-energy mode to determine the length of at least some of the bones of the skeleton. The single energy mode generally enables higher resolution images to be generated. The acquired images may then be used to measure bone length and determine changes in bone length from previous scans.

Various embodiments provide for measuring bone lengths using a dual-energy x-ray scan, for example a full body scan wherein all the bones of interest are imaged during a single scan, for example, a single imaging pass or operation. The lengths of different bones may be determined such as the long bones in the body, for example, the humerus and radius in the arm and the femur and tibia in the leg, among others. The total body scan may be acquired by different dual-energy imaging systems, for example, the Lunar iDXA imaging system available from GE Healthcare.

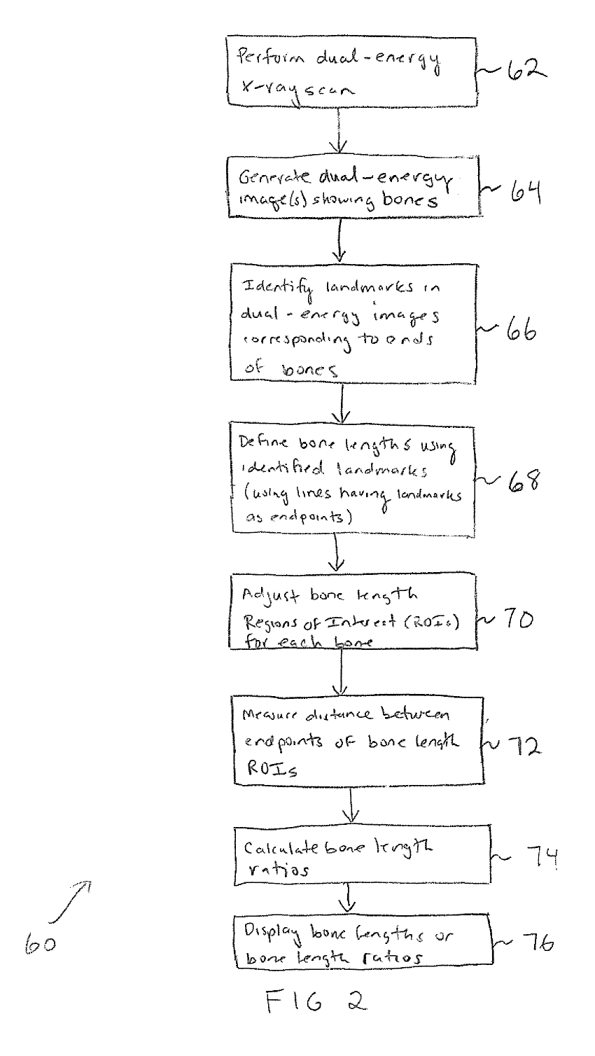

A method 60 for acquiring bone image information to determine the lengths of bones is shown in FIG. 2. The method 60 includes performing at 62 a dual-energy x-ray scan of an object, such as a patient or a portion of a patient. The patient in some embodiments lies supine on a table of a dual energy x-ray imaging system, such as a bone densitometer system. However, in other embodiments, the patient may be imaged with a bone densitometer system wherein the patient is imaged in a standing position or other position.

The dual-energy x-ray scan may be a rectilinear scan of the entire patient body, which may be performed in a raster-type scanning sequence as described in more detail herein. During the dual-energy x-ray scan an image of the entire skeleton of the patient may be acquired, which includes image information relating to the bones in the skeleton. The full body or total body scan of the entire body may be performed as a single scanning operation, which may be a low dose mode scan.

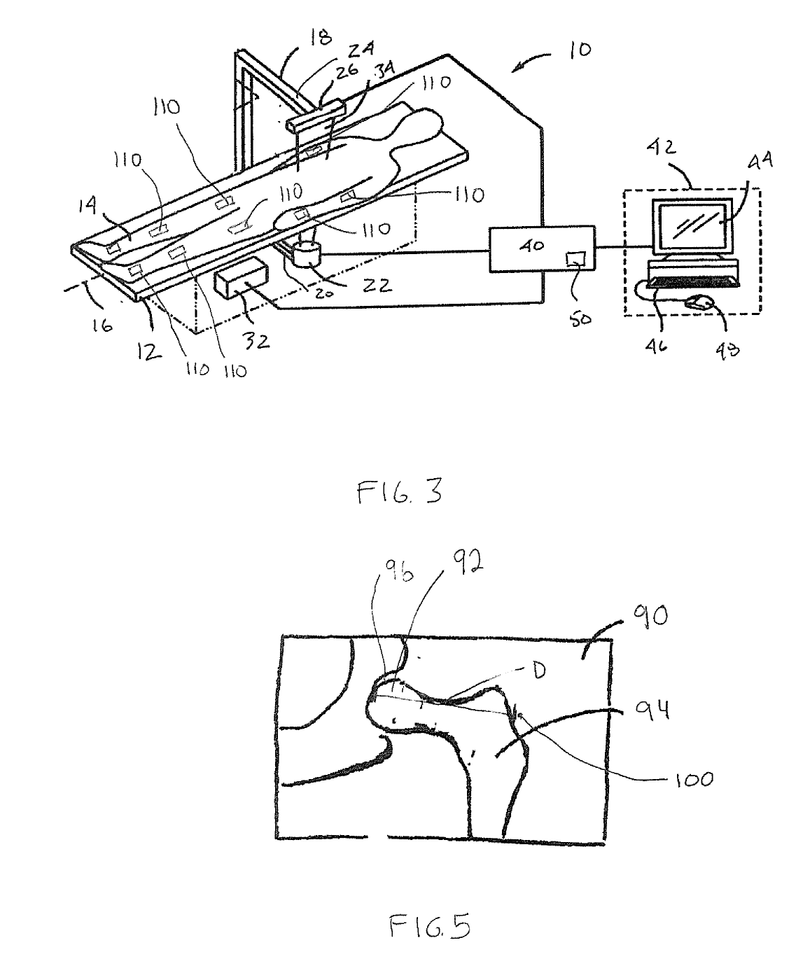

In some embodiments, instead of a full body or total body scan, individual rectangular scans of joints at the ends of all the bones of interest may be performed, which may be single sweep scans. For example, as shown in FIG. 3, wherein like numerals represent like parts with FIG. 1, individual rectangular scans 110 may be performed, which are each dual-energy x-ray scans. The individual rectangular scans 110 are performed at the ends of bones of interest, for example, at regions of the ends of bones of interest such that the end of the bones and joints are imaged. In some embodiments, ten rectangular scans 110 are performed to acquire end joint bone information with respect to the humerus and radius in each arm and the femur and tibia in each leg. These rectangular scans 10 result in less radiation dose than a full body scan. It should be noted that because of the location of the rectangular scans 110, namely that the location of the bone joint scans is known from the position of, for example, the c-arm, the length of the bone may be calculated from the end position of each of the bones near the joints based on the position of the c-arm. Accordingly, image information relating to the area between the joints, in particular, image information for the rest of the bone between the joints is not needed in this embodiment.

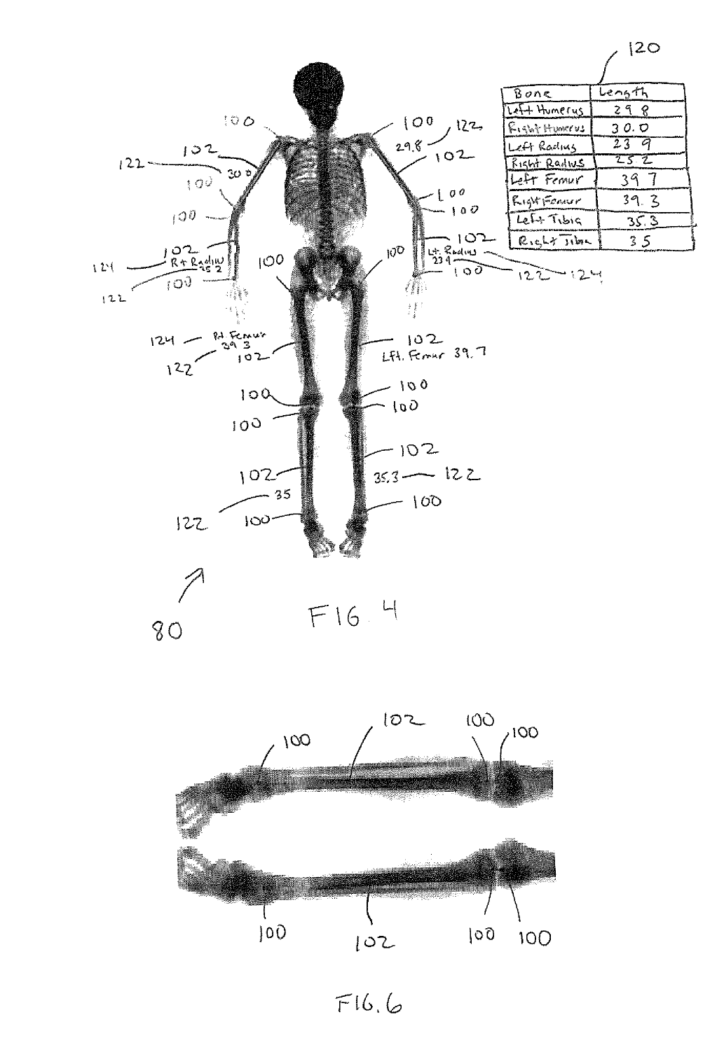

Referring again to the method 60, thereafter one or more dual-energy images are generated at 64 with each containing bone information, for example, bone image information and bone content information. Optionally, soft tissue information may be acquired from the dual-energy scan. For example, as shown in FIG. 4, a full body dual-energy image 80 may be generated from a scan of the entire body. Alternatively, one or more joint region dual-energy images 90 as shown in FIG. 5 may be generated, which is smaller than the full body dual-energy image 80. Accordingly, the full body dual-energy image 80 includes bone information (including bone content) and optionally soft tissue information regarding the skeletal structure over the entire scanned body. In the joint region dual-energy image 90, the bone and optional soft tissue information is provided for the region surrounding the joint 92 at the end of the bone 94, which in the illustrated embodiment is the joint 92 at the superior end of a femur bone at the patient's hip.

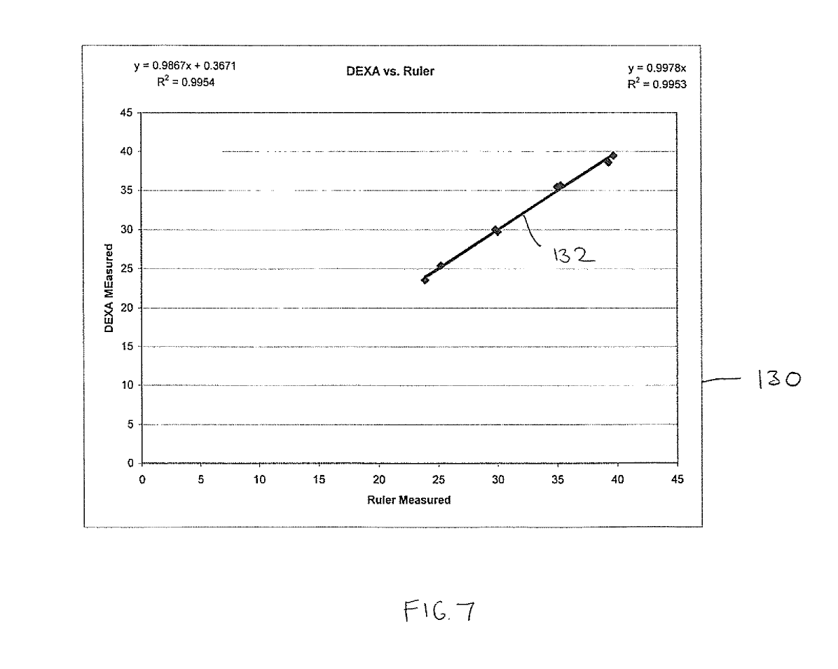

Using the full body dual-energy image 80 or the joint region dual-energy image 90 landmarks are identified at 66, and in particular, landmarks corresponding to the ends of the bones are identified. For example, fiducial marks are placed at the ends of the bones identifying the regions of interest. For example, one or more markers 100, such as points or marks are overlaid on the full body dual-energy image 80 or the joint region dual-energy image 90 corresponding to the end of the bone. However, it should be noted that any marking or indication may be provided to visually indicate the identified end of the bone. It also should be noted that the marker 100 may be placed adjacent a joint 92, for example, as shown in FIG. 5, and not on the joint 92 or in a gap 96 between two bones. For example, the marker 100 may be placed a distance D from the end of the joint 92 or the gap 96 between two bones, which distance may be predetermined based on the particular bone or may be determined based the contour or length of the bone, for example, following the contour. In general, the markers 100 may be positioned using any automatic or semi-automatic process known in the art in which fiducial points are identified on a bone, such as using a template structure or other methods as described herein. Alternatively, the markers 100 may be manually placed on full body dual-energy image 80 or the joint region dual-energy image 90 by an operator with a mouse and pointer. It also should be noted that the markers 100 may be placed on enlarged or zoomed in images, for example, the enlarged full body dual-energy image 80 or the joint region dual-energy image 90, or a portion thereof as shown in FIG. 6.

Once the ends of the bones are identified, and in particular, the endpoints of the bones are identified by the markers 100, bone lengths are defined at 68 using the identified landmark. Specifically, lines that have as endpoints the identified landmarks are used to determine the bone lengths. Accordingly, and for example, as shown in FIG. 4, for full body scans, lines 102 are defined between the markers 100 and which may be displayed on the full body dual-energy image 80 as colored lines. The regions of interest (ROIs), which are the lengths of the bones as defined by the lines 102, are initially placed along the length of the bones having as endpoints the markers 100. In some embodiments, the lines 102 defining the bone length ROIs are placed in the general vicinity of the bones of interest, for example, based on the position of the markers 100. It should be noted that in some embodiments, the markers 100 are not first positioned on the image(s), in which case only the lines 102 are generated with the lines ending at the identified landmarks determined at 66. It also should be noted that when joint region dual-energy images 90 are used, only the endpoints of the bones are identified with markers 100 and no lines 102 provided as the images are not of the entire bone length.

Thereafter, for each bone of interest, the bone length ROI may be adjusted at 70. For example, for each of the long bones of interest, the bone length ROI may be manually or automatically adjusted. During a manual adjustment process, an operator moves the line 102, moves the line 102 with the markers 100 or moves the markers 100, with the bone length ROI moved and displayed accordingly. For example, the operator may drag the entire line 102 or may move one or both of the markers 100, thereby defining one or two new endpoints for the bone length ROI. It should be noted that the adjustment performed at 70 may include changing not only the location of the markers 100 defining the endpoints and the direction of the line 102, but the length of the line 102. After each adjustment the operator may be prompted to confirm the new position of the bone length ROI. It also should be noted that not all the markers 100 have to be adjusted and only one marker 100 of a particular bone length ROI may be adjusted.

In other embodiments, the bone length ROI is adjusted automatically. However, the operator may thereafter also perform a manual adjustment as described above. The automatic adjustment may be performed in different manners. For example, the endpoint locations may be adjusted by convolving a kernel about a region around the identified end of the bone as marked by markers 100, for example, by an operator. In some embodiments, the endplates of the bones or gaps between bones may be identified with a kernel convolved about that region. The kernel may be any geometric or mathematical kernel, for example, based on a shape (template) or change in a parameter (e.g., change in a diameter of the bone) or a gap or spacing. Thus, the kernels may be used to adjust the location of the endpoints by determining a location of endplates of the bones in a region around the markers 100.

It should be noted that the kernel size and values are selected to pattern match the end of a particular bone of interest. It also should be noted that other bone information may be used to further refine the kernels, such as bone content information, for example, bone mineral content (BMC) and/or a bone mineral density (BMD) information.

Thereafter, the distance between the endpoints of the bone length ROIs is measured at 72. For example, the distances between the markers 100 along the lines 102, which may or may not have been adjusted are measured. In some embodiments, such as when the bone information is acquired from a full body dual-energy image 80, the bone length is calculated by determining a number of pixels along the bone length ROI between the endpoints. In particular, each pixel in the full body dual-energy image 80 has a known size in the vertical and horizontal direction. The length of the bone is calculated from the hypotenuse of the vertical distance in pixels between endpoints times the vertical sample size by the horizontal distance in pixels times the horizontal sample size. It should be noted that if the joint region dual-energy images 90 are used the calculation is the same, except the distance between the unscanned area between the endpoints is determined based on, for example, the distance traveled by the c-arm or imaging portion of the system between the rectangular scans. For example, the unscanned area between the endpoints may be determined as the distance traveled as registered by the translation controller or other motion controller of the dual-energy x-ray scanner.

It should be noted that in various embodiments, the lengths of different bones or bone combinations may be calculated based on the measured distance between the endpoints of the bone length ROIs. For example, in some embodiments, the lengths of the following bones and/or bone combinations are measured:

Femur (Left and/or Right)

Tibia (Left and/or Right)

Total Leg, i.e., Femur+Tibia (Left and/or Right)

Humerus (Left and/or Right)

Radius (Left and/or Right)

Total Arm, i.e., Humerus+Radius (Left and/or Right)

Arm Length Difference (Right Arm−Left Arm)

Leg Length Difference (Right Leg−Left Leg)

It should be noted that the lengths of different bones may be measured, and not just the bones described above or just long bones, which are merely described for illustrative purposes.

Referring again to the method 60, after the distance between the endpoints of the bone length ROIs are measured, such that the bone lengths are calculated, ratios of bone lengths may be calculated at 74. For example, the following ratios of bone lengths may be calculated:

Radius/Humerus (Left and/or Right)

Tibia/Femur (Left and/or Right)

Humerus/Femur (Left and/or Right)

Radius/Tibia (Left and/or Right)

It should be noted that different bone length ratios may be calculated other than the ratios described above, which are merely described for illustrative purposes.

Thereafter, one or more bone lengths or bone length ratios are displayed at 76. For example, the one or more bone lengths or bone length ratios are automatically displayed on a screen. It should be noted that the one or more bone lengths or bone length ratios may be displayed in a list format 120 (e.g., in columns and rows) or as length measurements indicators 122 or both as shown in FIG. 4, which length values are displayed in centimeters, but may be displayed in other units of measure, such as inches. It also should be noted that the order of the lengths listed may be modified, for example, all the left bone lengths may be listed followed by all the right bone lengths. When the length measurement indicators 122 are displayed, associated text 124 identifying the bone length being shown may or may not be displayed. It should be noted that bone length ratios are displayed in a similar manner.

Accordingly, as shown in the graph 130 of FIG. 7, the various embodiments provide bone measurements using dual-energy x-ray images that correlate with bone length measurements performed using a caliper (ruler). As can be seen from the line 132, the measurements correlate and the difference in bone lengths measured with the dual-energy x-ray images is less than 1 millimeter from the ruler measured bone length.

Thus, various embodiments of the invention provide for measurement of bones using dual-energy x-ray images, which may be acquired using a low dose mode of operation of a bone densitometer. The bone length measurements may be used in combination with other bone density and body composition measurements. In some embodiments, the measurements are determined from a single low dose full body scan. For example, using the Lunar iDXA bone densitometer, the following scan exposures in Table 1 are typical (dependent on body size):

TABLE 1

| Site | Typical Scan Exposure* |
|---|---|
| DVA | 329 μGy |
| Spine | 146 μGy |
| Femur | 146 μGy |
| DualFemur | 146 μGy |
| Forearm | 10 μGy |
| Total Body | 3 μGy |

Moreover, typical scan times using the Lunar iDXA bone densitometer (dependent on body size) are in Table 2 as follows:

TABLE 2

| Site | Typical Scan Time* |
|---|---|
| DVA | 2 min. |
| Spine | 30 s |
| Femur | 30 s |
| DualFemur | 60 s |
| Forearm | 20 s |
| Total Body | 4 min. |

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for measuring bone length, the method comprising:
acquiring bone image information from a dual-energy x-ray scan of a subject;
generating a dual-energy image using the acquired bone information; and
determining a length of at least one bone of the subject using the dual-energy image.

2. A method in accordance with claim 1 further comprising performing a full body dual-energy scan to acquire the bone information.

3. A method in accordance with claim 1 further comprising performing a rectangular dual-energy scan of a plurality of joint regions to acquire the bone information.

4. A method in accordance with claim 1 further comprising performing a rectilinear dual-energy scan to acquire the bone information.

5. A method in accordance with claim 1 further comprising performing a single dual-energy scan to acquire the bone information.

6. A method in accordance with claim 1 further comprising performing a dual-energy scan in a low dose mode to acquire the bone information.

7. A method in accordance with claim 1 further comprising performing a dual-energy scan in less than four minutes to acquire the bone information.

8. A method in accordance with claim 1 further comprising performing a dual-energy scan having less than three micro-Grays of absorbed radiation dose to acquire the bone information.

9. A method in accordance with claim 1 further comprising defining a bone length based on identified landmarks at endpoints of the bone.

10. A method in accordance with claim 9 wherein the identified landmark is operator defined.

11. A method in accordance with claim 9 wherein the identified landmark is automatically defined.

12. A method in accordance with claim 9 wherein the landmarks identify endplates of the bone.

13. A method in accordance with claim 12 further comprising adjusting a position of the identified landmark by convolving a kernel around the identified landmark.

14. A method in accordance with claim 1 wherein determining the length of the bone comprises measuring a distance between identified endpoints of the bone using a number of pixels in the dual-energy image between the endpoints.

15. A method in accordance with claim 14 wherein determining the length of the bone comprises using a determined location of a scanner that acquired the dual-energy image to determine a length of an unscanned portion of the bone.

16. A method in accordance with claim 1 further comprising calculating at least one bone length ratio based on a determined length of a plurality of bones.

17. A method in accordance with claim 1 further comprising displaying bone length information on a display based on the determined length of the bone.

18. A method for determining bone length, the method comprising:
acquiring image information with a bone densitometer using a dual-energy full body scan, the image information comprising bone content information and soft tissue information;
identifying endpoints of at least one bone using images formed from the image information; and
calculating a distance between the endpoints to determine a length of the at least one bone.

19. A method in accordance with claim 18 further comprising performing a single rectilinear low dose scan to acquire the image information.

20. A diagnostic imaging system comprising:
a dual-energy x-ray bone densitometer configured to acquire bone information from a dual-energy imaging scan of a subject; and
a bone length measurement module configured to determine a length of at least one bone of the subject using the bone information from the dual-energy imaging scan.
